(19) United States
(12) Patent Application Publication
(10) Pub. No.: US 2025/0352290 A1
(43) Pub. Date: Nov. 20, 2025

(54) SURGICAL INSTRUMENT WITH DUAL GRIP END EFFECTOR AND RELATED METHODS

(71) Applicant: Cilag GmbH International, Zug (CH)

(72) Inventors: Konstantin G. Zabotkin, Blue Ash, OH (US); Joseph M. Nath, West Chester, OH (US); Austin E. Wise, Cincinnati, OH (US); Karalyn R. Tellio, Cincinnati, OH (US)

(21) Appl. No.: 18/664,632

(22) Filed: May 15, 2024

(51) Int. Cl. A61B 34/37

(52) U.S. Cl. CPC A61B 34/37

(57) ABSTRACT

An apparatus includes a first jaw member including a first needle clamp pad and a first suture clamp pad. The apparatus also includes a second jaw member including a second needle clamp pad and a second suture clamp pad. The first and second jaw members are pivotable relative to each other between an open state, a first closed state in which the first and second suture clamp pads are configured to cooperate with each other to securely grip a suture, and a second closed state in which the first and second needle clamp pads are configured to cooperate with each other to securely grip a needle.

### BACKGROUND

A variety of surgical instruments include an end effector for use in conventional medical treatments and procedures conducted by a medical professional operator, as well as applications in robotically assisted surgeries. Such surgical instruments may be directly gripped and manipulated by a surgeon or incorporated into robotically assisted surgery. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletal gloves, master manipulators, or the like), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probe, etc. Each of these structures performs functions for the surgeon, for example, cutting tissue, coagulating tissue, holding or driving a needle, grasping a blood vessel, dissecting tissue, cauterizing tissue, and/or other functions.

As noted above, the surgeon may employ a needle driver to manipulate a needle. In some instances, the surgeon may desire to employ the same needle driver to manipulate a suture. However, the needle driver may not be suitable for manipulating the suture. For example, portions of the clamp pads of the needle driver, such as teeth, may undesirably crush or otherwise damage the suture. The dual mode needle drivers of the present disclosure seek to provide effective gripping of the needle when in a needle-gripping mode, as well as effective gripping of the suture when in a suture-gripping mode, while reducing or eliminating any risk of damaging the suture.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a first illustrative robotic system configured for a laparoscopic procedure;

FIG. 2 depicts a perspective view of a second illustrative robotic system;

FIG. 3 depicts an end elevational view of the robotic system of FIG. 2;

FIG. 4 depicts an end elevational view of the robotic system of FIG. 2 including an illustrative pair of robotic arms;

FIG. 5 depicts a partially exploded perspective view of the robotic arm of FIG. 4 having an instrument driver and a first illustrative surgical instrument;

FIG. 6A depicts a side elevational view of the surgical instrument of FIG. 5 in a retracted position;

FIG. 6B depicts a side elevational view the surgical instrument of FIG. 5 in an extended position;

FIG. 7A depicts an enlarged perspective view of a second illustrative surgical instrument, more particularly a grasper instrument;

FIG. 7B depicts another enlarged perspective view of the grasper instrument of FIG. 7A, with a distal clevis of the grasper instrument illustrated as transparent to show certain internal features thereof;

FIG. 7C depicts a side elevational view of the grasper instrument of FIG. 7A;

FIG. 7D depicts another side elevational view of the grasper instrument of FIG. 7A;

FIG. 7E depicts a top plan view of a proximal clevis of the grasper instrument of FIG. 7A;

FIG. 8 depicts an enlarged perspective view of a third illustrative surgical instrument, more particularly, a needle driver instrument;

FIG. 9 depicts a perspective view of a dual mode needle driver of the needle driver instrument of FIG. 8;

FIG. 10 depicts a side elevational view of a distal end of a jaw of the dual mode needle driver of FIG. 9;

FIG. 11 depicts a top plan view of the distal end of the jaw of FIG. 10;

FIG. 12 depicts a partially exploded perspective view of the distal end of the jaw of FIG. 10;

FIG. 13A depicts a side elevational view of the dual mode needle driver of FIG. 9, showing the dual mode needle driver in an open state;

FIG. 13B depicts a side elevational view of the dual mode needle driver of FIG. 9, showing the dual mode needle driver in a first closed state such that the dual mode needle driver is in a suture-gripping mode;

FIG. 13C depicts a side elevational view of the dual mode needle driver of FIG. 9, showing the dual mode needle driver in a second closed state such that the dual mode needle driver is in a needle-gripping mode;

FIG. 14A depicts a side elevational view of an illustrative input device, more particularly, a handle, for use with the dual mode needle driver instrument of FIG. 8, showing the handle in an open position; and

FIG. 14B depicts a side elevational view of the handle of FIG. 14A, showing the handle in a closed position.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “side,” “upwardly,” and “downwardly” also are used herein for reference to relative positions and directions. Such terms are used below with reference to views as illustrated for clarity and are not intended to limit the invention described herein.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved ease of use such that one or more of the instruments of the robotically-enabled medical system may be controlled by a single operator.

### I. Example of Robotically-Enabled Medical System

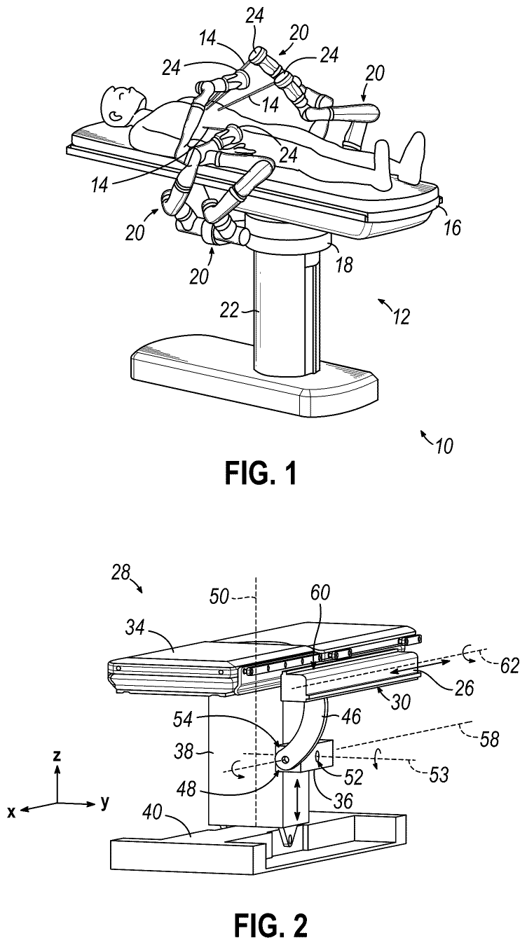

FIG. 1 shows an example of a robotically-enabled medical system, including a first example of a robotic system (10). Robotic system (10) of the present example includes a table system (12) operatively connected to a surgical instrument (14) for a diagnostic and/or therapeutic procedure in the course of treating a patient. Such procedures may include, but are not limited, to bronchoscopy, ureteroscopy, a vascular procedure, and a laparoscopic procedure. To this end, surgical instrument (14) is configured for a laparoscopic procedure, although it will be appreciated that any instrument for treating a patient may be similarly used. At least part of robotic system (10) may be constructed and operable in accordance with at least some of the teachings of any of the various patents, patent application publications, and patent applications that are cited herein.

### A. Example of Robotic System with Annular Carriage

As shown in FIG. 1, robotic system (10) includes table system (12) having a platform, such as a table (16), with a plurality of carriages (18) which may also be referred to herein as “arm supports,” respectively supporting the deployment of a plurality of robotic arms (20). Robotic system (10) further includes a support structure, such as a column (22), for supporting table (16) over the floor. Table (16) may also be configured to tilt to a desired angle during use, such as during laparoscopic procedures. Each robotic arm (20) includes an instrument driver (24) configured to removably connect to and manipulate surgical instrument (14) for use. In alternative examples, instrument drivers (24) may be collectively positioned in a linear arrangement to support the instrument extending therebetween along a “virtual rail” that may be repositioned in space by manipulating the one or more robotic arms (20) into one or more angles and/or positions. In practice, a C-arm (not shown) may be positioned over the patient for providing fluoroscopic imaging.

In the present example, column (22) includes carriages (18) arranged in a ring-shaped form to respectively support one or more robotic arms (20) for use. Carriages (18) may translate along column (22) and/or rotate about column (22) as driven by a mechanical motor (not shown) positioned within column (22) in order to provide robotic arms (20) with access to multiples sides of table (16), such as, for example, both sides of the patient. Rotation and translation of carriages (18) allows for alignment of instruments, such as surgical instrument (14), into different access points on the patient. In alternative examples, such as those discussed below in greater detail, robotic system (10) may include a surgical bed with adjustable arm supports including a bar (26) (see FIG. 2) extending alongside. One or more robotic arms (20) may be attached to carriages (18) (e.g., via a shoulder with an elbow joint). Robotic arms (20) are vertically adjustable so as to be stowed compactly beneath table (16), and subsequently raised during use.

Robotic system (10) may also include a tower (not shown) that divides the functionality of robotic system (10) between table (16) and the tower to reduce the form factor and bulk of table (16). To this end, the tower may provide a variety of support functionalities to table (16), such as computing and control capabilities, power, fluidics, optical processing, and/or sensor data processing. The tower may also be movable so as to be positioned away from the patient to improve medical professional access and de-clutter the operating room. The tower may also include a master controller or console that provides both a user interface for operator input, such as keyboard and/or pendant, as well as a display screen, including a touchscreen, for pre-operative and intra-operative information, including, but not limited to, real-time imaging, navigation, and tracking information. In some versions, the tower may include gas tanks to be used for insufflation.

### B. Example of Robotic System with Bar Carriage

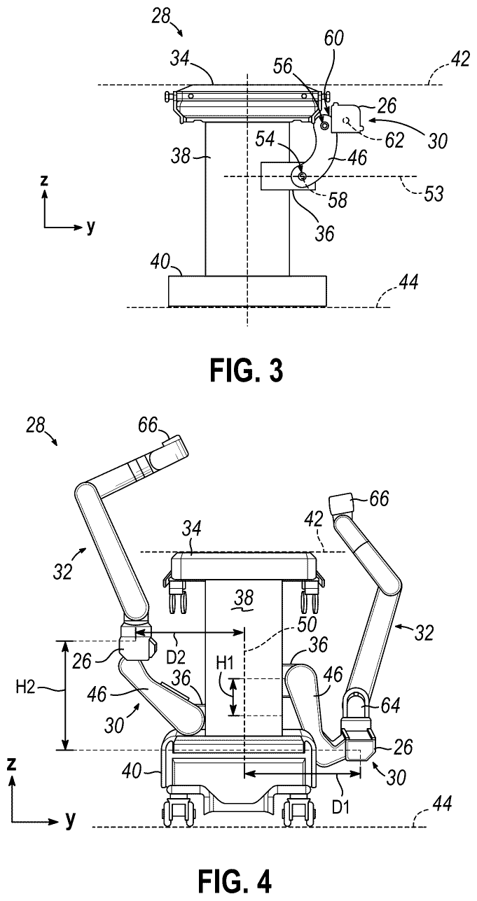

FIGS. 2-4 show another example of a robotic system (28). Robotic system (28) of this example includes one or more adjustable arm supports (30) including bars (26) that are configured to support one or more robotic arms (32) relative to a table (34). In the present example, a single adjustable arm support (30) (FIGS. 2-3) and a pair of adjustable arm supports (30) (FIG. 4) are shown, though additional arm supports (30) may be provided about table (34). Each adjustable arm support (30) is configured to selectively move relative to table (34) so as to alter the position of adjustable arm support (30), and/or any robotic arms (32) mounted thereto, relative to table (34) as desired. Such adjustable arm supports (30) may provide high versatility to robotic system (28), including the ability to easily stow one or more adjustable arm supports (30) with robotic arms (32) beneath table (34).

Each adjustable arm support (30) provides several degrees of freedom, including lift, lateral translation, tilt, etc. In the present example shown in FIGS. 2-4, arm support (30) is configured with four degrees of freedom, which are illustrated with arrows. A first degree of freedom allows adjustable arm support (30) to move in the z-direction (“Z-lift”). For example, adjustable arm support (30) includes a vertical carriage (36). Vertical carriage (36) is configured to move up or down along or relative to a column (38) and a base (40), both of which support table (34). A second degree of freedom allows adjustable arm support (30) to tilt about an axis extending in the y-direction. For example, adjustable arm support (30) includes a rotary joint, which allows adjustable arm support (30) to align with table (34) when table (34) is in a Trendelenburg position or other inclined position. A third degree of freedom allows adjustable arm support (30) to “pivot up” about an axis extending in the x-direction, which may be useful to adjust a distance between a side of table (34) and adjustable arm support (30). A fourth degree of freedom allows translation of adjustable arm support (30) along a longitudinal length of table (34), which extends along the x-direction. Base (40) and column (38) together support table (34) relative to a support surface, which is shown along a support axis (42) above a floor axis (44) in the present example. While the present example shows adjustable arm support (30) mounted to column (38), arm support (30) may alternatively be mounted to table (34) or base (40).

As shown in the present example, adjustable arm support (30) includes vertical carriage (36), a bar connector (46), and bar (26). To this end, vertical carriage (36) attaches to column (38) by a first joint (48), which allows vertical carriage (36) to move relative to column (38) (e.g., such as up and down a first, vertical axis (50) extending in the z-direction). First joint (48) provides the first degree of freedom (“Z-lift”) to adjustable arm support (30). Adjustable arm support (30) further includes a second joint (52), which provides the second degree of freedom (tilt) for adjustable arm support (30) to pivot about a second axis (53) extending in the y-direction. Adjustable arm support (30) also includes a third joint (54), which provides the third degree of freedom (“pivot up”) for adjustable arm support (30) about a third axis (58) extending in the x-direction. Furthermore, an additional joint (56) mechanically constrains third joint (54) to maintain a desired orientation of bar (26) as bar connector (46) rotates about third axis (58). Adjustable arm support (30) includes a fourth joint (60) to provide a fourth degree of freedom (translation) for adjustable arm support (30) along a fourth axis (62) extending in the x-direction.

FIG. 4 shows a version of robotic system (28) with two adjustable arm supports (30) mounted on opposite sides of table (34). A first robotic arm (32) is attached to one such bar (26) of first adjustable arm support (30). This first robotic arm (32) includes a connecting portion (64) attached to a first bar (26). Similarly, a second robotic arm (32) includes connecting portion (64) attached to the other bar (26). As shown in FIG. 4, vertical carriages (36) are separated by a first height (H1), and bar (26) is disposed a second height (H2) from base (40). The first bar (26) is disposed a first distance (D1) from vertical axis (50), and the other bar (26) is disposed a second distance (D2) from vertical axis (50). Distal ends of first and second robotic arms (32) respectively include instrument drivers (66), which are configured to attach to one or more instruments such as those discussed below in greater detail.

In some versions, one or more of robotic arms (32) has seven or more degrees of freedom. In some other versions, one or more robotic arms (32) has eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and connecting portion (64) (1-degree of freedom including translation). In some versions, the insertion degree of freedom is provided by robotic arm (32); while in some other versions, an instrument such as surgical instrument includes an instrument-based insertion architecture.

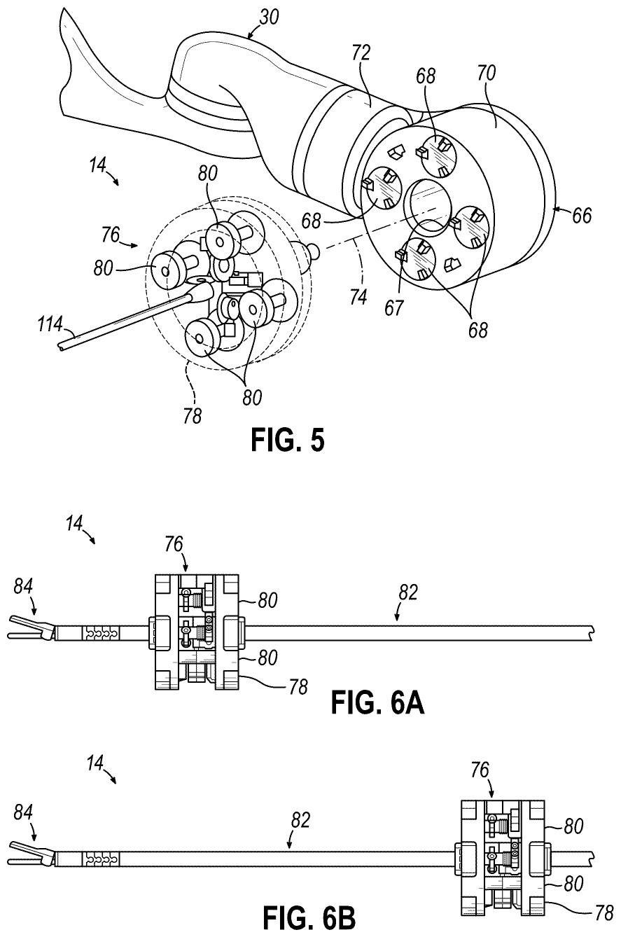

FIG. 5 shows one example of instrument driver (66) in greater detail, with surgical instrument (14) removed therefrom. Given the present instrument-based insertion architecture shown with reference to surgical instrument (14), instrument driver (66) further includes a clearance bore (67) extending entirely therethrough so as to movably receive a portion of surgical instrument (14) as discussed below in greater detail. Instrument driver (66) may also be referred to herein as an “instrument drive mechanism,” an “instrument device manipulator,” or an “advanced device manipulator” (ADM). Instruments may be configured to be detached, removed, and interchanged from instrument driver (66) for individual sterilization or disposal by the medical professional or associated staff. In some scenarios, instrument drivers (66) may be draped for protection and thus may not need to be changed or sterilized.

Each instrument driver (66) operates independently of other instrument drivers (66) and includes a plurality of rotary drive outputs (68), such as four drive outputs (68), also independently driven relative to each other for directing operation of surgical instrument (14). Instrument driver (66) and surgical instrument (14) of the present example are aligned such that the axes of each drive output (68) are parallel to the axis of surgical instrument (14). In use, control circuitry (not shown) receives a control signal, transmits motor signals to desired motors (not shown), compares resulting motor speed as measured by respective encoders (not shown) with desired speeds, and modulates motor signals to generate desired torque at one or more drive outputs (68).

In the present example, instrument driver (66) is circular with respective drive outputs (68) housed in a rotational assembly (70). In response to torque, rotational assembly (70) rotates along a circular bearing (not shown) that connects rotational assembly (70) to a non-rotational portion (72) of instrument driver (66). Power and controls signals may be communicated from non-rotational portion (72) of instrument driver (66) to rotational assembly (70) through electrical contacts therebetween, such as a brushed slip ring connection (not shown). In one example, rotational assembly (70) may be responsive to a separate drive output (not shown) integrated into non-rotatable portion (72), and thus not in parallel to the other drive outputs (68). In any case, rotational assembly (70) allows instrument driver (66) to rotate rotational assembly (70) and drive outputs (68) in conjunction with surgical instrument (14) as a single unit around an instrument driver axis (74).

### C. Example of Surgical Instrument with Instrument-based Insertion Architecture

FIGS. 5-6B show surgical instrument (14) having the instrument-based insertion architecture as discussed above. Surgical instrument (14) includes an elongated shaft assembly (82), an end effector (84) connected to and extending distally from shaft assembly (82), and an instrument base (76) (shown with a transparent external skin for discussion purposes) coupled to shaft assembly (82). Instrument base (76) includes an attachment surface (78) and a plurality of drive inputs (80) (such as receptacles, pulleys, and spools) configured to receive and couple with respective rotary drive outputs (68) of instrument driver (66). Insertion of shaft assembly (82) is grounded at instrument base (76) such that end effector (84) is configured to selectively move longitudinally from a retracted position (FIG. 6A) to an extended position (FIG. 6B), vice versa, and any desired longitudinal position therebetween. As used herein, the retracted position is shown in FIG. 6A and places end effector (84) relatively close and proximally toward instrument base (76); whereas the extended position is shown in FIG. 6B and places end effector (84) relatively far and distally away from instrument base (76). Insertion into and withdrawal of end effector (84) relative to the patient may thus be facilitated by surgical instrument (14), although it will be appreciated that such insertion into and withdrawal may also occur via adjustable arm supports (30) in one or more examples.

When coupled to rotational assembly (70) of instrument driver (66), surgical instrument (14), comprising instrument base (76) and instrument shaft assembly (82), rotates in combination with rotational assembly (70) about the instrument driver axis (74). Since instrument shaft assembly (82) is positioned at the center of instrument base (76), instrument shaft assembly (82) is coaxial with instrument driver axis (74) when attached. Thus, rotation of the rotational assembly (70) causes instrument shaft assembly (82) to rotate about its own longitudinal axis. Moreover, as instrument base (76) rotates with instrument shaft assembly (82), any tendons connected to drive inputs (80) of instrument base (76) are not tangled during rotation. Accordingly, the parallelism of the axes of rotary drive outputs (68), rotary drive inputs (80), and instrument shaft assembly (82) allows for the shaft rotation without tangling any control tendons, and clearance bore (67) provides space for translation of shaft assembly (82) during use.

The foregoing examples of surgical instrument (14) and instrument driver (66) are merely illustrative examples. Robotic arms (32) may interface with different kinds of instruments in any other suitable fashion using any other suitable kinds of interface features. Similarly, different kinds of instruments may be used with robotic arms (32), and such alternative instruments may be configured and operable differently from surgical instrument (14).

In addition to the foregoing, robotic systems (10, 28) may be configured and operable in accordance with at least some of the teachings of U.S. Pat. No. 9,737,371, entitled “Configurable Robotic Surgical System with Virtual Rail and Flexible Endoscope,” issued Aug. 22, 2017, the disclosure of which is incorporated by reference herein, in its entirety; U.S. Pat. No. 10,945,904, entitled “Tilt Mechanisms for Medical Systems and Applications,” issued Mar. 16, 2021, the disclosure of which is incorporated by reference herein, in its entirety; U.S. Pub. No. 2019/0350662, entitled “Controllers for Robotically-Enabled Teleoperated Systems,” published Nov. 21, 2019, the disclosure of which is incorporated by reference herein, in its entirety; U.S. Pub. No. 2020/0085516, entitled “Systems and Methods for Concomitant Medical Procedures,” published Mar. 19, 2020; and/or U.S. Pub. No. 2021/0401527, entitled “Robotic Medical Systems Including User Interfaces with Graphical Representations of User Input Devices,” published Dec. 30, 2021, the disclosure of which is incorporated by reference herein, in its entirety.

### II. Surgical Instrument with Grasper

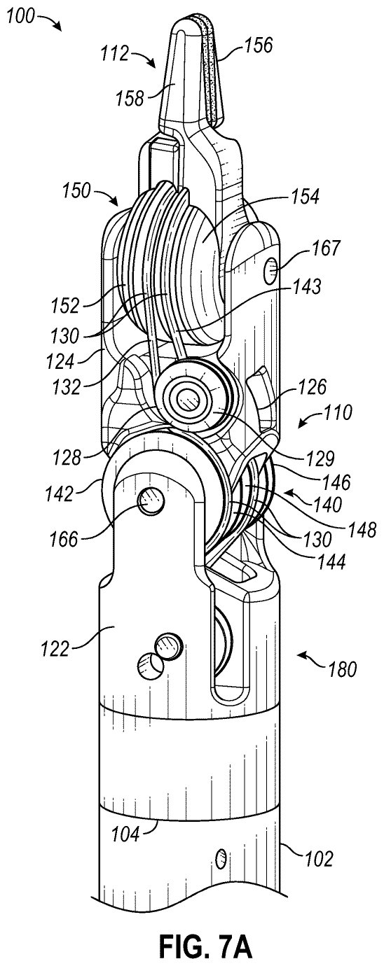

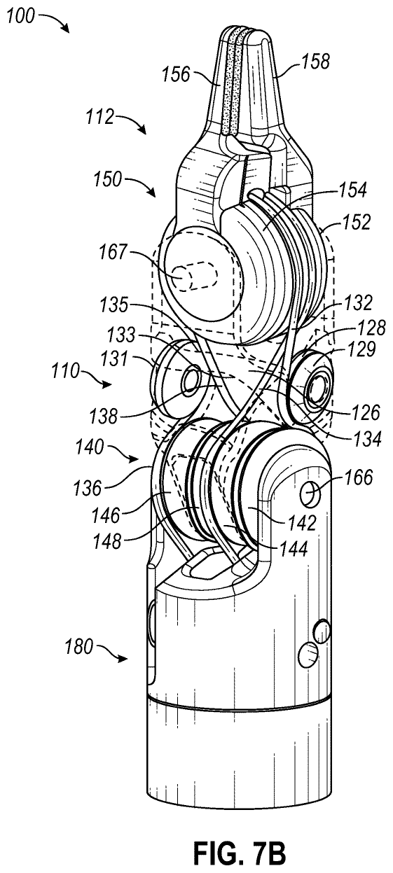

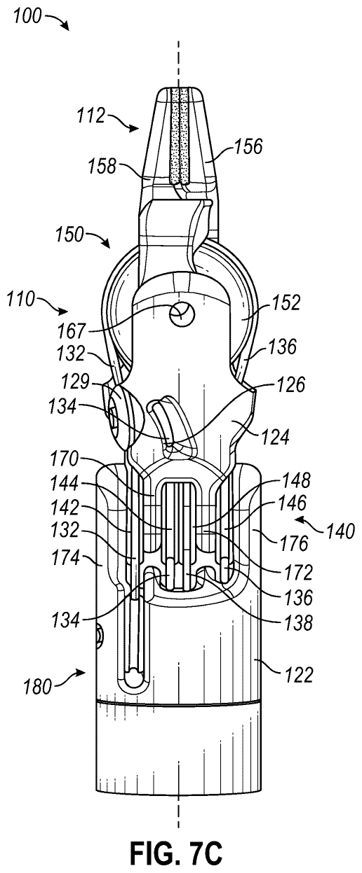

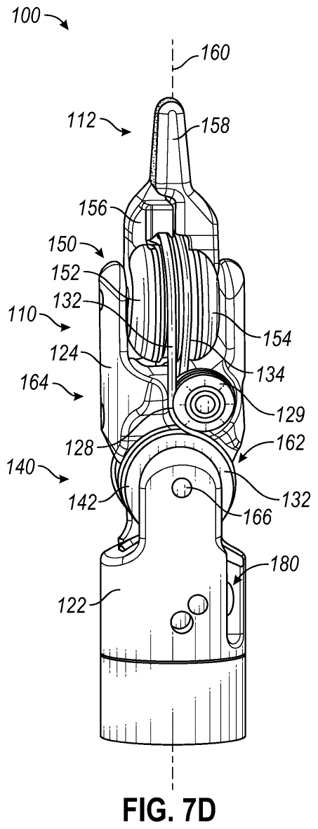

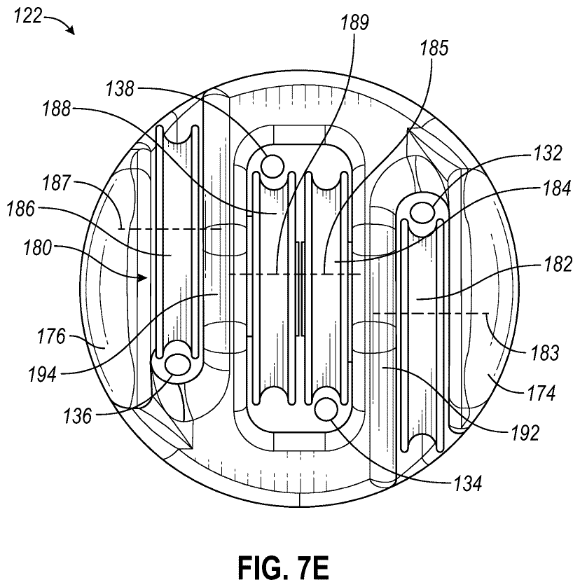

FIGS. 7A-7E illustrate an example of a medical instrument (100) that may be incorporated into a robotic medical system, such as either of the robotic systems (10, 28) described above. For example, instrument (100) may be readily incorporated into either robotic system (10) in place of any of instruments (14).

FIG. 7A is a perspective view of the medical instrument (100). FIG. 7B is another perspective view of the medical instrument (100), shown with a distal clevis (124) illustrated as transparent so as to visualize certain internal features thereof. FIG. 7C is a first side view of the medical instrument (100). FIG. 7D is a second side view of the medical instrument (100). FIG. 7E is a top view of a proximal clevis (122) of the medical instrument (100).

As shown in FIG. 7A, in the illustrated embodiment, the medical instrument (100) includes an elongated shaft (102) extending to a distal end (104). A wrist (110) is positioned at the distal end (104) of the elongated shaft (102). The wrist (110) is also connected to an end effector (112), which is a grasper in the illustrated embodiment.

In the illustrated embodiment, the wrist (110) comprises a proximal clevis (122) and a distal clevis (124). The proximal clevis (122) can be attached to the distal end (104) of the elongated shaft (102). In the illustrated embodiment, the distal clevis (124) is pivotally attached to the proximal clevis (122) by an axle (166) which extends through the distal clevis (124) and the proximal clevis (122). The distal clevis (124) can rotate about an axis of the axle (166) relative to the proximal clevis (122).

As best seen in FIG. 7C, the proximal clevis (122) can include a first proximal clevis support leg (174) and a second proximal clevis support leg (176). The axle (166) can extend through the first proximal clevis support leg (174) and the second proximal clevis support leg (176) of the proximal clevis (122). Similarly, the distal clevis (124) can include a first distal clevis support leg (170) and a second distal clevis support leg (172). The axle (166) extends through the first distal clevis support leg (170) and the second distal clevis support leg (172) of the distal clevis (124).

As shown in FIGS. 7A-7D, the medical instrument (100) includes a plurality of proximal pulleys (140) and a plurality of distal pulleys (150) positioned in the wrist (110). As best seen in FIGS. 7A-7C, the proximal pulleys (140) can be positioned on the axle (166) that connects the proximal clevis (122) and the distal clevis (124). In the illustrated embodiment, the proximal pulleys (140) include a first outer proximal pulley (142), a first inner proximal pulley (144), a second outer proximal pulley (146), and a second inner proximal pulley (148). The first outer proximal pulley (142), the first inner proximal pulley (144), the second outer proximal pulley (146), and the second inner proximal pulley (148) can each be positioned on the axle (166) such that they can rotate about the axle (166).

As seen in FIGS. 7A-7D, the distal pulleys (150) can be positioned on an axle (167). The axle (167) can extend through the distal clevis (124) as shown. In the illustrated embodiment, the distal pulleys (150) include a first distal pulley (152) and a second distal pulley (154) mounted on the axle (167).

The pitch axle (166) and the yaw axle (167) can be oriented at an angle with respect to each other. In the illustrated example, the pitch axle (166) and the yaw axle (167) are orthogonal. Accordingly, the pitch plane and the yaw plane can also be orthogonal to each other.

The end effector (112) of the medical instrument (100) can be formed by a first jaw member (156) and a second jaw member (158). The first jaw member (156) can be connected to the first distal pulley (152) and the second jaw member (158) can be connected to the second distal pulley (154). The orientation of the end effector (112) can be controlled by rotating the first distal pulley (152) and the second distal pulley (154) in the same direction about the axle (167). For example, by rotating both of the first distal pulley (152) and the second distal pulley (154) in the same direction about the axle (167), the yaw of the end effector (112) can be adjusted. The end effector (112) can be actuated (e.g., opened or closed in the case of the illustrated grasper) by rotating the first distal pulley (152) and the second distal pulley (154) in the opposite directions about the axle (167).

The medical instrument (100) can include a plurality of pull wires (130) that can be actuated (e.g., pulled or tensioned) to control the three degrees of freedom of the medical instrument (100) (pitch, yaw, and actuation). As shown in FIGS. 7A-7D, the plurality of pull wires (130) are engaged with the proximal pulleys (140) and the distal pulleys (150). In the illustrated embodiment, the plurality of pull wires (130) include a first pull wire segment (132), a second pull wire segment (134), a third pull wire segment (136), and a fourth pull wire segment (138) which are routed along various paths through the wrist (110).

For example, in the illustrated embodiment, the first pull wire segment (132) engages the first outer proximal pulley (142) and the first distal pulley (152). Actuation of the first pull wire segment (132) can be associated with closing the first jaw member (156). The second pull wire segment (134) can be engaged with the first inner proximal pulley (144) and the second distal pulley (154). The second pull wire segment (134) can be associated with opening the second jaw member (158). The third pull wire segment (136) can be engaged with the second outer proximal pulley (146) and second distal pulley (154). The third pull wire segment (136) can be associated with closing the second jaw member (158). The fourth pull wire segment (138) can be engaged with the second inner proximal pulley (148) and the first distal pulley (152). The fourth pull wire segment (138) can be associated with opening the first jaw member (156).

As shown in the figures, each of the first pull wire segment (132) and the fourth pull wire segment (138) can engage the first distal pulley (152), but on opposite sides. Similarly, each of the second pull wire segment (134) and the third pull wire segment (136) can engage the second distal pulley (154), but on opposite sides. In the illustrated embodiment, each of the proximal pulleys (140) is only engaged by one of the pull wire segments. The first pull wire segment (132) engages the first outer proximal pulley (142) on the same side of the wrist (110) that the fourth pull wire segment (138) engages the second inner proximal pulley (148). Similarly, the second pull wire segment (134) engages the first inner proximal pulley (144) on the same side of the wrist (110) that the third pull wire segment (136) engages the second outer, proximal pulley (146). At the proximal pulleys (140), the first and fourth pull wire segments (132, 138) are positioned on an opposite side of the wrist (110) than the second and third pull wire segments (134, 136).

As best seen in FIG. 7B, which illustrates the distal clevis (124) as transparent, the plurality of pull wires (130) are redirected between proximal pulleys (140) and distal pulleys (150). To accomplish the redirection, the wrist (110) of the instrument (100) includes hybrid redirect surfaces. Specifically, in the illustrated embodiment, the wrist (110) includes a pair of static redirect surfaces and a pair of dynamic redirect surfaces positioned between proximal pulleys (140) and distal pulleys (150). As shown in FIG. 7B, the pair of static redirect surfaces include a first static redirect surface (126) and a second static redirect surface (133). The first static redirect surface (126) and the second static redirect surface (133) can each be an angled or curved surface formed in or on the distal clevis (124). An example is visible in FIG. 7C, which shows the static redirect surface (126). The pair of dynamic redirect surfaces include a first dynamic redirect surface (128) and a second dynamic redirect surface (131). Each of the first dynamic redirect surface (128) and the second dynamic redirect surface (131) can comprise a surface of a redirect pulley, such as the first redirect pulley (129) and the second redirect pulley (135) that are illustrated in the figures.

The plurality of pull wires (130) are redirected by the static redirect surfaces (126, 133) and the dynamic redirect surfaces (128, 131). In the illustrated embodiment, the first pull wire segment (132) engages the first dynamic redirect surface (128). The second pull wire segment (134) engages the first static redirect surface (126). The third pull wire segment (136) engages the second dynamic redirect surface (131). The fourth pull wire segment (138) engages the second static redirect surface (133).

Thus, in this example, the first and third pull wire segments (132, 136), which are associated with closing the end effector (112) are redirected using the dynamic redirect surfaces (128, 131) of the redirect pulleys (129, 135), respectively. The second and fourth pull wire segments (134, 138), which are associated with opening the end effector (112) are redirected using the static redirect surfaces (126, 133), respectively.

The medical instrument (100) also includes shaft redirect pulleys (180) positioned in the proximal clevis (122) and/or within the elongated shaft (102). The shaft redirect pulleys (180) are best seen in FIG. 7E which is a top down view of the proximal clevis (122). As shown, the shaft redirect pulleys (180) include a first outer shaft redirect pulley (182), a first inner shaft redirect pulley (184), a second outer shaft redirect pulley (186), and second inner shaft redirect pulley (188). In the illustrated embodiment, the shaft redirect pulleys (180) are in a staggered position. That is, as shown in FIG. 7E, the first outer shaft redirect pulley (182) is positioned on first axis (183) and the first inner shaft redirect pulley (184) is positioned on second axis (185). The first and second axes (183, 185) are not coaxial (in the illustrated embodiment). The second inner shaft redirect pulley (188) is positioned on a third axis (189). In the illustrated embodiment the third axis (189) is coaxial with second axis (185). The second outer shaft redirect pulley (186) is positioned on fourth axis (187). In the illustrated embodiment, the fourth axis (187) is not coaxial with the first, second, or third axes (183, 185, 189). The proximal clevis (122) also comprises a first proximal clevis support wall (192) and a second proximal clevis support wall (194). The first proximal clevis support wall (192) is positioned between the first inner and outer shaft redirect pulleys (182, 184). The second proximal clevis support wall (194) is positioned between the second inner and outer shaft redirect pulleys (186, 184). The first proximal clevis support leg (174) and the second proximal clevis support leg (176) are also shown in FIG. 7E.

By way of further example, medical instrument (100) may be configured and operable in accordance with at least some of the teachings of U.S. Pub. No. 2020/0405423, entitled “Medical Instruments Including Wrists with Hybrid Redirect Surfaces,” published Dec. 31, 2020, the disclosure of which is incorporated by reference herein, in its entirety.

### III Surgical Instrument with Dual Mode Needle Driver

In some instances, it may be desirable to configure medical instrument (100) as a needle driver instrument that is capable of providing effective gripping of both a needle and a suture. For example, it may be desirable to configure medical instrument (100) as a dual mode needle driver instrument that is capable of providing effective gripping of the needle when in a needle-gripping mode, as well as effective gripping of the suture when in a suture-gripping mode, while reducing or eliminating any risk of damaging the suture.

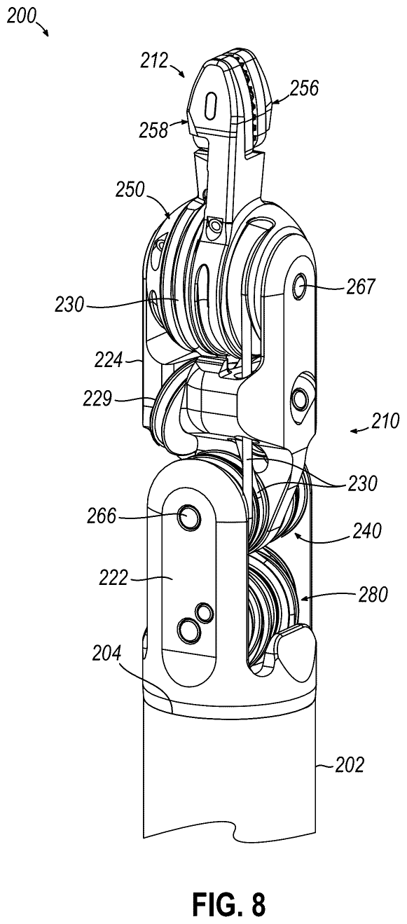

FIGS. 8-13C show a portion of an example of a medical instrument (200) that may provide such functionality. Medical instrument (200) may be similar to medical instrument (100) described above, except as otherwise described below. In this regard, medical instrument (200) may be incorporated into a robotic medical system, such as either of the robotic systems (10, 28) described above. For example, instrument (200) may be readily incorporated into either robotic system (10) in place of any of instruments (14). Instrument (200) of the present example includes an elongated shaft (202) extending along a longitudinal axis to a distal end (204), a wrist (210) positioned at distal end (204), and an end effector in the form of a dual mode needle driver (212) connected to wrist (210).

As shown in FIG. 8, wrist (210) comprises a proximal clevis (222) and a distal clevis (224) similar to proximal clevis (122) and distal clevis (224) described above, respectively. In this regard, proximal clevis (222) is attached to distal end (204) of elongated shaft (202), and distal clevis (224) is pivotally attached to proximal clevis (222) by a pitch axle (266) such that distal clevis (224) can rotate about an axis of axle (266) relative to proximal clevis (222). Medical instrument (200) also includes a plurality of proximal pulleys (240) and a plurality of distal pulleys (250) similar to proximal pulleys (140) and distal pulleys (150) described above, respectively. In this regard, proximal pulleys (240) are positioned on axle (266), and distal pulleys (250) are positioned on a yaw axle (267).

In the example shown, dual mode needle driver (212) of medical instrument (200) includes a first jaw member (256) and a second jaw member (258) connected to respective distal pulleys (250) in a manner similar to that described above in connection with first and second jaw members (156, 158), such that dual mode needle driver (212) can be actuated (e.g., opened or closed) by rotating the respective distal pulleys (250) in opposite directions about axle (267).

Medical instrument (200) also includes a plurality of pull wires (230) similar to pull wires (130) described above. In this regard, pull wires (230) can be actuated (e.g., pulled or tensioned) to control the three degrees of freedom of medical instrument (200) (pitch, yaw, and actuation). As shown in FIG. 8, the plurality of pull wires (230) are engaged with proximal pulleys (240) and distal pulleys (250), with various segments of pull wires (230) routed along various paths through wrist (210) in a manner similar to that described above in connection with pull wires (130). In the example shown, the plurality of pull wires (230) are redirected between proximal pulleys (240) and distal pulleys (250) via one or more dynamic redirect surfaces defined by one or more redirect pulleys (229) (one shown), which may be similar to redirect pulleys (129, 135) described above, and/or via one or more static redirect surfaces (not shown), which may be similar to static redirect surfaces (126, 133) described above. Medical instrument (200) also includes shaft redirect pulleys (280) positioned in proximal clevis (222) and/or within elongated shaft (202), similar to shaft redirect pulleys (180) described above.

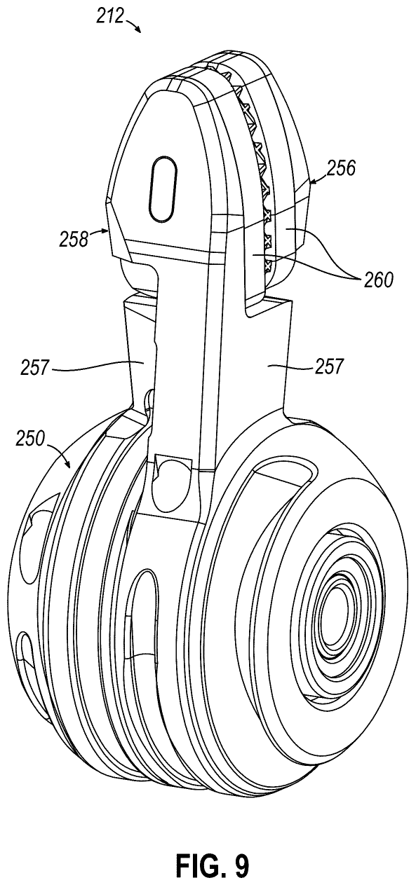

Referring primarily to FIGS. 9-12, each jaw member (256, 258) of dual mode needle driver (212) includes a clamp arm (257) extending distally relative to the respective distal pulley (250) and having a distal, laterally-inwardly facing surface (259). Jaw members (256, 258) are arranged such that distal, laterally-inwardly facing surfaces (259) are configured to be opposed from each other when jaw members (256, 258) are pivoted toward each other to define at least one closed state of dual mode needle driver (212).

Each jaw member (256, 258) further includes a needle clamp pad (260) coupled to the distal, laterally-inwardly facing surface (259) of the respective clamp arm (257). In the example shown, each needle clamp pad (260) includes a laterally-inwardly facing surface (261) and a plurality of rigid, substantially pyramid-shaped teeth (262) extending laterally inwardly therefrom. While teeth (262) of the present example are each substantially pyramid-shaped, it will be appreciated that one or more teeth (262) may have any other suitable shape(s). For example, teeth (262) may each be substantially cylindrical, substantially triangular, substantially conical, and/or may collectively define a substantially undulating profile.

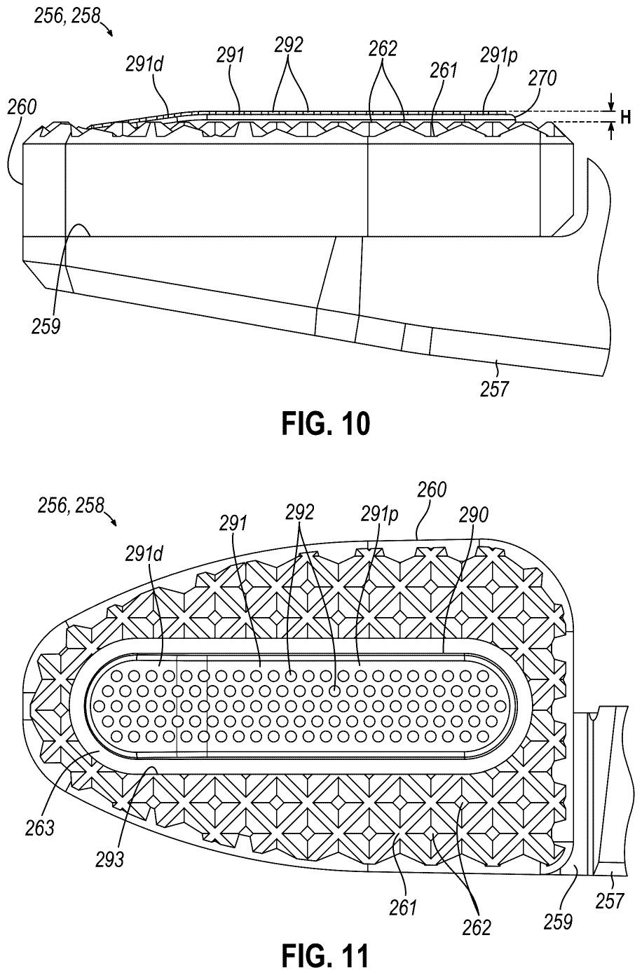

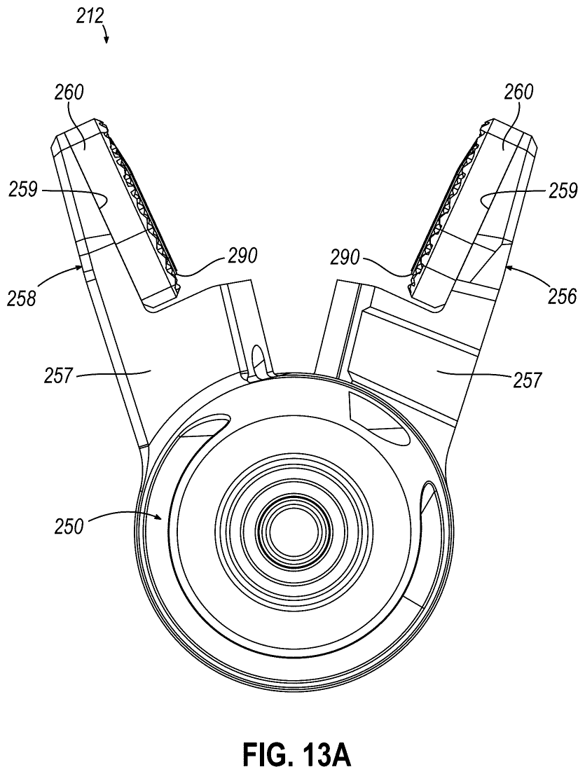

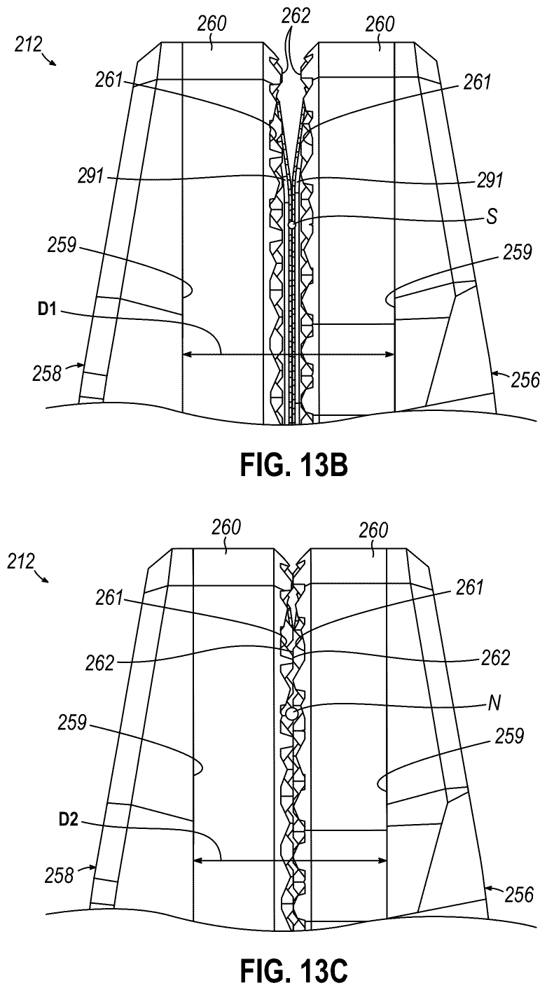

As shown in FIG. 10, the laterally-inwardly facing surface (261) of each needle clamp pad (260) of the present example is substantially parallel to the distal, laterally-inwardly facing surface (259) of the respective clamp arm (257). Laterally-inwardly facing surfaces (261) are configured to confront each other when jaw members (256, 258) are pivoted toward each other to define the at least one closed state of dual mode needle driver (212). In addition, or alternatively, the teeth (262) of needle clamp pads (260) may be configured to confront and/or mesh (e.g., interlock) with each other when jaw members (256, 258) are pivoted toward each other to define the at least one closed state of dual mode needle driver (212). In some versions, each needle clamp pad (260), including the respective teeth (262), may be formed of a metal material. When jaw members (256, 258) are pivoted toward each other to define the at least one closed state of dual mode needle driver (212), teeth (262) of first jaw member (256) may be configured to cooperate with teeth (262) of second jaw member (258) to securely grip a needle (N) (FIG. 13C), for example. Due to the rigidity of teeth (262), such gripping of needle (N) by teeth (262) may be referred to as a “hard grip.”

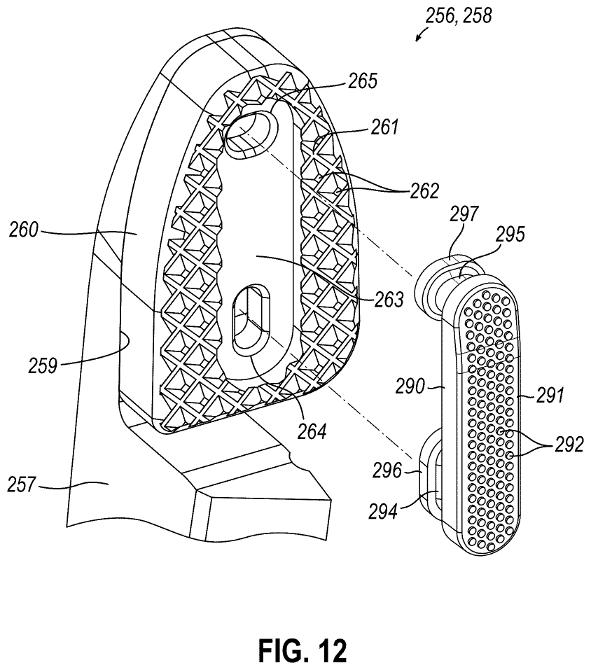

As shown in FIGS. 11 and 12, each needle clamp pad (260) of the present example includes an elongate recess (263) extending laterally outwardly from the respective laterally-inwardly facing surface (261), with teeth (262) being arranged about the respective elongate recess (263) such that each elongate recess (263) is substantially surrounded by the corresponding teeth (262). As shown in FIG. 12, each needle clamp pad (260) of the present example also includes a proximal bore (264) and a distal bore (265) each extending laterally outwardly from the respective elongate recess (263) to a laterally-outwardly facing surface of the respective needle clamp pad (260).

In the example shown, each jaw member (256, 258) also includes a suture clamp pad (290) coupled to the corresponding needle clamp pad (260). In the example shown, each suture clamp pad (290) includes a laterally-inwardly facing surface (291) and a plurality of flexible, substantially cylindrical protrusions (292) extending laterally inwardly therefrom. While protrusions (292) of the present example are each substantially cylindrical, it will be appreciated that one or more protrusions (292) may have any other suitable shape(s). For example, protrusions (292) may each be substantially pyramid-shaped, substantially triangular, substantially conical, and/or may collectively define a substantially undulating profile.

As shown in FIG. 10, the laterally-inwardly facing surface (291) of each suture clamp pad (290) of the present example includes a proximal portion (291p) that is substantially parallel to the laterally-inwardly facing surface (261) of the respective needle clamp pad (260), and a distal portion (291d) that tapers laterally outwardly from the respective proximal portion (291p) toward a distal end of the respective suture clamp pad (290). In some other versions, distal portion (291d) may be substantially parallel to the laterally-inwardly facing surface (261) of the respective needle clamp pad (260). As shown, at least the protrusions (292) on proximal portions (291p) of laterally-inwardly facing surfaces (291) are each positioned laterally inwardly relative to the teeth (262) of the respective needle clamp pad (260) such that protrusions (292) are spaced apart from the teeth (262) of the respective needle clamp pad (260) by a height (H) (e.g., within the frame of reference of FIG. 10), at least when the respective suture clamp pad (290) is in an undeformed (e.g., uncompressed) state. In this manner, at least a portion of each suture clamp pad (290) may be proud relative to the corresponding needle clamp pad (260), at least when in the undeformed state. Laterally-inwardly facing surfaces (291) (e.g., at least proximal portions (291p) thereof) are configured to confront and/or deform (e.g., compress) each other when jaw members (256, 258) are pivoted toward each other to define the at least one closed state of dual mode needle driver (212). In addition, or alternatively, the protrusions (292) of suture clamp pads (290) may be configured to confront and/or deform (e.g., compress) each other when jaw members (256, 258) are pivoted toward each other to define the at least one closed state of dual mode needle driver (212). Each suture clamp pad (290), including the respective protrusions (292), may be formed of a soft, flexible, pliable, and/or compressible material. In some versions, each suture clamp pad (290), including the respective protrusions (292), may be formed of a polymeric (e.g., elastomeric) material, such as silicone. When jaw members (256, 258) are pivoted toward each other to define the at least one closed state of dual mode needle driver (212), surface (291) and/or protrusions (292) of first jaw member (256) may be configured to cooperate with surface (291) and/or protrusions (292) of second jaw member (258) to securely grip a suture(S) (FIG. 13B), for example. Due to the flexibility of surfaces (291) and protrusions (292), such gripping of needle (N) by protrusions (292) may be referred to as a “soft grip.”

As shown in FIG. 11, each suture clamp pad (290) of the present example is securely retained within the elongate recess (263) of the respective needle clamp pad (260), such that each suture clamp pad (290) is substantially surrounded by the corresponding teeth (262). Each suture clamp pad (290) may thereby define an island of flexible material relative to the rigid material of the respective needle clamp pad (260). In the example shown, an outer periphery of each suture clamp pad (290) is spaced apart from an inner periphery of the elongate recess (263) of the respective needle clamp pad (260) by a generally elongated, annular relief channel (293), at least when the respective suture clamp pad (290) is in an undeformed state, such that each relief channel (293) may be configured to receive corresponding portions of the respective suture clamp pad (290) when the respective suture clamp pad (290) is in a deformed (e.g., compressed) state. As shown in FIG. 12, each suture clamp pad (290) of the present example also includes a proximal peg (294) and a distal peg (295) each extending laterally outwardly from a laterally-outwardly facing surface of the respective suture clamp pad (290) and terminating at respective laterally outer flanges (296, 297). Proximal and distal pegs (294, 295) are received within the proximal and distal bores (264, 265) of the respective needle clamp pad (260), respectively, for securely coupling the respective suture clamp pad (290) to the respective needle clamp pad (260). In some versions, each suture clamp pad (290) may be overmolded onto the respective needle clamp pad (260), with pegs (294, 295) and the corresponding flanges (296, 297) being formed by molten material flowing through the respective bores (264, 265) during the overmolding process.

While each suture clamp pad (290) of the example shown is substantially surrounded by the corresponding teeth (262) to thereby define an island of flexible material relative to the rigid material of the respective needle clamp pad (260), it will be appreciated that other versions may have an inverse arrangement. For example, the teeth (262) of each needle clamp pad (260) may be substantially surrounded by the corresponding suture clamp pad (290) to thereby define an island of rigid material relative to the flexible material of the corresponding suture clamp pad (290). In addition, or alternatively, dual mode needle driver (212) may be self-righting so as to orient needle (N) in a predetermined manner (e.g., vertically) in instances where needle clamp pads (260) are not aligned. In some versions, only one of jaw members (256, 258) may be equipped with a suture clamp pad (290).

Referring now to FIGS. 13A-13C, in an example of a method of use, dual mode needle driver (212) may initially be in an open state in which jaw members (256, 258) are pivoted away from each other, such as the open state shown in FIG. 13A. While dual mode needle driver (212) is in the open state, an object, such as needle (N) or suture(S), may be positioned between jaw members (256, 258) to facilitate subsequent manipulating of needle (N) or suture(S) by dual mode needle driver (212).

As shown in FIG. 13B, with suture(S) positioned between jaw members (256, 258), jaw members (256, 258) may be pivoted toward each other to define a first closed (e.g., partially closed) state of dual mode needle driver (212) in which dual mode needle driver (212) provides a soft grip of suture(S), such that dual mode needle driver (212) is in a suture-gripping mode. More particularly, jaw members (256, 258) may be pivoted toward each other to position laterally-inwardly facing surfaces (259) at a first distance (D1) from each other.

When laterally-inwardly facing surfaces (259) are at the first distance (D1) from each other, surface (291) and/or protrusions (292) of first jaw member (256) may cooperate with surface (291) and/or protrusions (292) of second jaw member (258) to securely grip suture(S); while the first distance (D1) may be sufficiently large to prevent teeth (262) of jaw members (256, 258) from engaging suture(S). In some instances, surface (291) and/or protrusions (292) of first jaw member (256) may bear against surface (291) and/or protrusions (292) of second jaw member (258) sufficiently to cause slight deformation (e.g., compression) of one or both suture clamp pads (290) when laterally-inwardly facing surfaces (259) are at the first distance (D1) from each other, such that height (H) may slightly decrease for one or both suture clamp pads (290). However, such slight deformation of one or both suture clamp pads (290) and such corresponding slight decrease of height (H) may be insufficient to permit engagement of teeth (262) with suture(S). For example, despite such slight deformation of one or both suture clamp pads (290), teeth (262) may remain laterally outward relative to the corresponding surface (291) and/or protrusions (292). Thus, when in the suture-gripping mode, dual mode needle driver (212) may provide the soft grip of suture(S) via suture clamp pads (290) while preventing teeth (262) from crushing or otherwise damaging suture(S).

As shown in FIG. 13C, with needle (N) positioned between jaw members (256, 258), jaw members (256, 258) may be pivoted toward each other to define a second closed (e.g., fully closed) state of dual mode needle driver (212) in which dual mode needle driver (212) provides a hard grip of needle (N), such that dual mode needle driver (212) is in a needle-gripping mode. More particularly, jaw members (256, 258) may be pivoted toward each other to position laterally-inwardly facing surfaces (259) at a second distance (D2) from each other that is less than the first distance (D1).

When laterally-inwardly facing surfaces (259) are at the second distance (D2) from each other, teeth (262) of first jaw member (256) may be configured to cooperate with teeth (262) of second jaw member (258) to securely grip needle (N). In this regard, surface (291) and/or protrusions (292) of first jaw member (256) may bear against surface (291) and/or protrusions (292) of second jaw member (258) sufficiently to cause substantial deformation (e.g., compression) of both suture clamp pads (290) when laterally-inwardly facing surfaces (259) are at the second distance (D2) from each other, such that height (H) may substantially decrease (e.g., to zero and/or less than zero) for one or both suture clamp pads (290). In this manner, portions of each suture clamp pad (290) may be flush with and/or recessed relative to the corresponding needle clamp pad (260). In some instances, each relief channel (293) may receive corresponding portions of the respective suture clamp pad (290) to accommodate such substantial deformation of the respective suture clamp pad (290). Such substantial deformation of both suture clamp pads (290) and such corresponding substantial decrease of height (H) may be sufficient to permit engagement of teeth (262) with needle (N). For example, due to such substantial deformation of both suture clamp pads (290), suture clamp pads (290) may be sufficiently compressed out of the path of teeth (262) to permit teeth (262) of first jaw member (256) to mesh (e.g., interlock) with teeth (262) of second jaw member (258), and to further permit teeth (262) to engage needle (N). Thus, when in the needle-gripping mode, dual mode needle driver (212) may provide the hard grip of needle (N) via needle clamp pads (260).

While dual mode needle driver (212) is shown transitioning from the open state shown in FIG. 13A, to the first closed state shown in FIG. 13B, and then to the second closed state shown in FIG. 13C, dual mode needle driver (212) may be transitioned between the open and closed states in any suitable order.

It will therefore be appreciated that dual mode needle driver (212) is capable of providing effective gripping of needle (N) when in the needle-gripping mode, as well as effective gripping of suture(S) when in the suture-gripping mode, and that dual mode needle driver (212) may reduce or eliminate any risk of damaging the suture(S) when in the suture-gripping mode. In some instances, the use of flexible suture clamp pads (290) to grip suture(S) may allow dual mode needle driver (212) to cradle suture(S) within the deformed portions of suture clamp pads (290) and thereby reduce reliance on frictional engagement between suture(S) and suture clamp pads (290) to grip suture(S). In addition, or alternatively, the use of flexible suture clamp pads (290) to grip suture(S) may allow dual mode needle driver (212) to manipulate suture(S) while applying a reduced clamping force to suture(S) relative to that which might otherwise be required if rigid needle clamp pads (260) were used to grip suture(S). Thus, such use of flexible suture clamp pads (290) may reduce the amount of tension applied to the corresponding pull wires (230) for closing jaw members (256, 258) to grip suture(S). This may, in turn, extend the useful life of the corresponding pull wires (230).

As noted above, medical instrument (200) may be incorporated into either of the robotic systems (10, 28) described above in place of any of instruments (14). As also noted above, either of robotic systems (10, 28) may include a controller (not shown) that provides a user interface for operator input. In some instances, it may be desirable for such a controller to provide audible and/or haptic feedback to the operator that is indicative of whether dual mode needle driver (212) is in the needle-gripping mode or the suture-gripping mode.

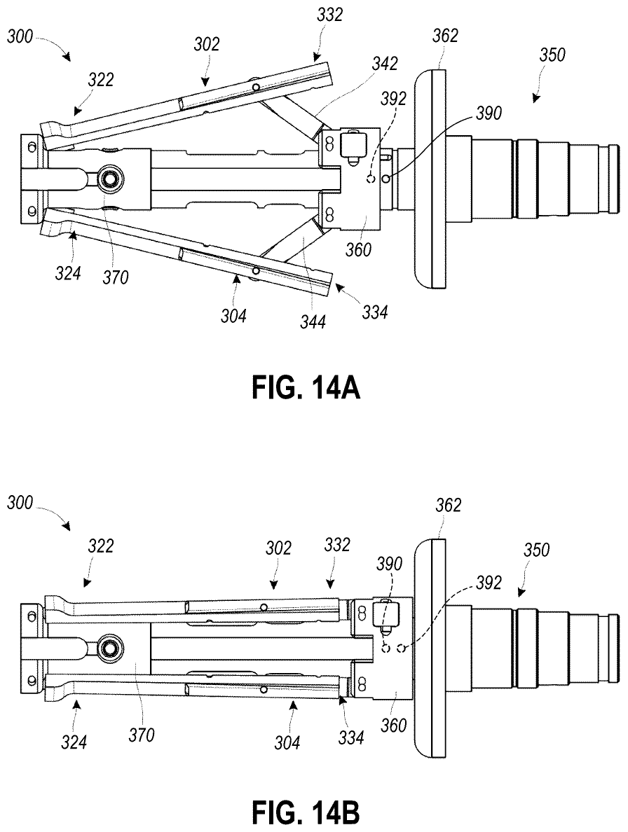

FIGS. 14A-14B show an example of a hand input device of a controller in the form of a handle (300) that may provide such functionality. Handle (300) may be configured and operable in accordance with at least some of the teachings of U.S. Pat. No. 11,207,147, entitled “Hand-Manipulated Input Device for Robotic System,” issued Dec. 28, 2021, the disclosure of which is incorporated by reference herein, in its entirety. In this regard, handle (300) of the present example includes first and second links (302, 304) having respective distal ends (322, 324) and respective proximal ends (332, 334). As shown, links (302, 304) are connected at their respective distal ends (322, 324) to a distal link support (370), and are connected at their respective proximal ends (332, 334) to a sliding support (360) with secondary links (342, 344), respectively. Secondary links (342, 344) may be free to pivot to change an angular displacement of the respective link (302, 304) into an axial translation of sliding support (360) along a central shaft (350). In the open position shown in FIG. 14A, sliding support (360) is positioned more distally along central shaft (350); in the closed position shown in FIG. 14B, sliding support (360) is positioned more proximally along central shaft (350). Handle (300) also includes a proximal plate (362) that is attached to or integral with the central support shaft (350) to limit axial translation of sliding support (360).

Links (302, 304) can be maneuvered in a pinching motion, which can be translated to pivoting of jaw members (256, 258) toward each other. Each link (302, 304) may be biased in an open position. In some configurations, each link can be spring-loaded in an open position. In some configurations, there are at least two springs for each link (302, 304) with a first spring providing the majority of the force to bias the link (302, 304) in an open position. A second spring can provide haptic feedback when the link (302, 304) reaches a certain degree of closure to indicate to the operator that dual mode needle driver (212) is in the suture-gripping mode, and that further motion to close handle (300) will result in dual mode needle driver (212) transitioning into the needle-gripping mode.

In the example shown, handle (300) further includes at least one detent (390, 392) configured to provide audible and/or haptic feedback to the operator that is indicative of whether dual mode needle driver (212) is in the needle-gripping mode or the suture-gripping mode. More particularly, handle (300) of the present example includes a first detent (390) extending outwardly from central support shaft (350), and a second detent (392) extending inwardly from sliding support (360) such that detents (390, 392) are configured to selectively engage each other during axial translation of sliding support (360) and thereby generate the audible and/or haptic feedback. For example, detents (390, 392) may be positioned relative to each other such that detents (390, 392) engage each other when dual mode needle driver (212) transitions from the suture-gripping mode into the needle-gripping mode. Thus, the operator may readily determine that dual mode needle driver (212) is in the suture-gripping mode while pinching links (302, 304) prior to receiving the audible and/or haptic feedback generated by the engagement between detents (390, 392); and may readily determine that dual mode needle driver (212) is in the needle-gripping mode while pinching links (302, 304) after receiving the audible and/or haptic feedback generated by the engagement between detents (390, 392).

In some instances, either of robotic systems (10, 28) may include a surgical visualization system that is configured and operable in accordance with at least some of the teachings of U.S. Pat. No. 10,925,598, entitled “Robotically-Assisted Surgical Suturing Systems,” issued Feb. 23, 2021, the disclosure of which is incorporated by reference herein, in its entirety. Such a surgical visualization system may include an imaging device configured to enable the identification of suture(S) and/or needle (N), such as through spectral analysis, photo-acoustics, and/or ultrasound, for example. In some cases, a controller may be configured to automatically adjust the clamping force applied by dual mode needle driver (212) based on the identification of suture(S) and/or needle (N) between jaw members (256, 258) by such a surgical visualization system. For example, the controller may be configured to automatically transition dual mode needle driver (212) into the suture-gripping mode in response to the surgical visualization system identifying suture(S) between jaw members (256, 258); and/or may be configured to automatically transition dual mode needle driver (212) into the needle-gripping mode in response to the surgical visualization system identifying needle (N) between jaw members (256, 258).

In some instances, the controller may be configured to bypass commands received via handle (300) based on the identification of suture(S) and/or needle (N) between jaw members (256, 258) by the surgical visualization system. For example, the controller may be configured to ignore pinching of links (302, 304) beyond the engagement between detents (390, 392) in response to the surgical visualization system identifying suture(S) between jaw members (256, 258), to thereby prevent dual mode needle driver (212) from transitioning from the suture-gripping mode into the needle-gripping mode.

### IV. Illustrative Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a first jaw member including: (i) a first needle clamp pad, and (ii) a first suture clamp pad; and (b) a second jaw member including: (i) a second needle clamp pad, and (ii) a second suture clamp pad, the first and second jaw members being pivotable relative to each other between: (i) an open state, (ii) a first closed state in which the first and second suture clamp pads are configured to cooperate with each other to securely grip a suture, and (iii) a second closed state in which the first and second needle clamp pads are configured to cooperate with each other to securely grip a needle.

### Example 2

The apparatus of Example 1, wherein the first and second needle clamp pads are configured to be spaced apart from each other when the first and second jaw members are in the first closed state.

### Example 3

The apparatus of any of Examples 1 through 2, wherein the first and second suture clamp pads are configured to deform each other when the first and second jaw members are in the second closed state.

### Example 4

The apparatus of any of Examples 1 through 3, wherein the first and second needle clamp pads are rigid.

### Example 5

The apparatus of Example 4, wherein the first and second needle clamp pads comprise metal.

### Example 6

The apparatus of any of Examples 1 through 5, wherein the first and second suture clamp pads are flexible.

### Example 7

The apparatus of Example 6, wherein the first and second suture clamp pads comprise at least one elastomer.

### Example 8

The apparatus of any of Examples 1 through 7, wherein the first and second suture clamp pads are overmolded onto the first and second needle clamp pads, respectively.

### Example 9

The apparatus of any of Examples 1 through 8, wherein the first and second suture clamp pads are proud relative to the first and second needle clamp pads, respectively, when the first and second jaw members are in each of the open state and the first closed state.

### Example 10

The apparatus of any of Examples 1 through 9, wherein the first and second suture clamp pads are at least one of flush with or recessed relative to the first and second needle clamp pads, respectively, when the first and second jaw members are in the second closed state.

### Example 11

The apparatus of any of Examples 1 through 10, wherein the first and second suture clamp pads are substantially surrounded by respective portions of the first and second needle clamp pads, respectively.

### Example 12

The apparatus of any of Examples 1 through 11, wherein the first and second needle clamp pads include first and second recesses, respectively, wherein the first and second suture clamp pads are securely retained within the first and second recesses, respectively.

### Example 13

The apparatus of any of Examples 1 through 12, wherein the first and second needle clamp pads each include a plurality of rigid teeth.

### Example 14

The apparatus of any of Examples 1 through 13, wherein the first and second suture clamp pads each include a plurality of flexible protrusions.

### Example 15

The apparatus of any of Examples 1 through 14, wherein the first closed state is a partially closed state, wherein the second closed state is a fully closed state.

### Example 16

An apparatus, comprising: (a) a shaft extending along a longitudinal axis to a distal end; and (b) an end effector operatively coupled to the distal end of the shaft, the end effector including a pair of jaw members, the pair of jaw members being pivotable relative to each other for gripping at least one surgical object, at least one jaw member of the pair of jaw members including: (i) a first clamp pad, and (ii) a second clamp pad configured to transition between an undeformed state and a deformed state, the second clamp pad of the at least one jaw member extending laterally inwardly relative to the respective first clamp pad at least when in the undeformed state.

### Example 17

The apparatus of Example 16, wherein the first clamp pad of the at least one jaw member is rigid.

### Example 18

The apparatus of any of Examples 16 through 17, further comprising a wrist, wherein the end effector is operatively coupled to the distal end of the shaft via the wrist.

### Example 19

A system, comprising: (a) an end effector configured to transition between a suture-gripping mode in which the end effector is configured to apply a soft grip to a suture, and a needle-gripping mode in which the end effector is configured to apply a hard grip to a needle; and (b) a controller configured to transition the end effector between the suture-gripping mode and the needle-gripping mode, wherein the controller is configured to provide at least one of audible feedback or haptic feedback to indicate transitioning of the end effector from the suture-gripping mode to the needle-gripping mode.

### Example 20

The system of Example 19, wherein the controller includes at least one detent configured to generate the at least one of audible feedback or haptic feedback during transitioning of the end effector between the suture-gripping mode and the needle-gripping mode.

### V. Miscellaneous

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Some versions of the examples described herein may be implemented using a processor, which may be part of a computer system and communicate with a number of peripheral devices via bus subsystem. Versions of the examples described herein that are implemented using a computer system may be implemented using a general-purpose computer that is programmed to perform the methods described herein. Alternatively, versions of the examples described herein that are implemented using a computer system may be implemented using a specific-purpose computer that is constructed with hardware arranged to perform the methods described herein. Versions of the examples described herein may also be implemented using a combination of at least one general-purpose computer and at least one specific-purpose computer.

In versions implemented using a computer system, each processor may include a central processing unit (CPU) of a computer system, a microprocessor, an application-specific integrated circuit (ASIC), other kinds of hardware components, and combinations thereof. A computer system may include more than one type of processor. The peripheral devices of a computer system may include a storage subsystem including, for example, memory devices and a file storage subsystem, user interface input devices, user interface output devices, and a network interface subsystem. The input and output devices may allow user interaction with the computer system. The network interface subsystem may provide an interface to outside networks, including an interface to corresponding interface devices in other computer systems. User interface input devices may include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

In versions implemented using a computer system, a storage subsystem may store programming and data constructs that provide the functionality of some or all of the modules and methods described herein. These software modules may be generally executed by the processor of the computer system alone or in combination with other processors. Memory used in the storage subsystem may include a number of memories including a main random-access memory (RAM) for storage of instructions and data during program execution and a read only memory (ROM) in which fixed instructions are stored. A file storage subsystem may provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem in the storage subsystem, or in other machines accessible by the processor.

In versions implemented using a computer system, the computer system itself may be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the example of the computer system described herein is intended only as a specific example for purposes of illustrating the technology disclosed. Many other configurations of a computer system are possible having more or fewer components than the computer system described herein.

As an article of manufacture, rather than a method, a non-transitory computer readable medium (CRM) may be loaded with program instructions executable by a processor. The program instructions when executed, implement one or more of the computer-implemented methods described above. Alternatively, the program instructions may be loaded on a non-transitory CRM and, when combined with appropriate hardware, become a component of one or more of the computer-implemented systems that practice the methods disclosed.

Versions described above may be designed to be disposed of after a single use, or they can be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof are placed in a closed and sealed container, such as a plastic or TYVEK bag. The container and system, instrument, and/or portion thereof may then be placed in a field of radiation that can penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) a first jaw member including:
(i) a first needle clamp pad, and
(ii) a first suture clamp pad; and
(b) a second jaw member including:
(i) a second needle clamp pad, and
(ii) a second suture clamp pad,
the first and second jaw members being pivotable relative to each other between:
(i) an open state,
(ii) a first closed state in which the first and second suture clamp pads are configured to cooperate with each other to securely grip a suture, and
(iii) a second closed state in which the first and second needle clamp pads are configured to cooperate with each other to securely grip a needle.

2. The apparatus of claim 1, wherein the first and second needle clamp pads are configured to be spaced apart from each other when the first and second jaw members are in the first closed state.

3. The apparatus of claim 1, wherein the first and second suture clamp pads are configured to deform each other when the first and second jaw members are in the second closed state.

4. The apparatus of claim 1, wherein the first and second needle clamp pads are rigid.

5. The apparatus of claim 4, wherein the first and second needle clamp pads comprise metal.

6. The apparatus of claim 1, wherein the first and second suture clamp pads are flexible.

7. The apparatus of claim 6, wherein the first and second suture clamp pads comprise at least one elastomer.

8. The apparatus of claim 1, wherein the first and second suture clamp pads are overmolded onto the first and second needle clamp pads, respectively.

9. The apparatus of claim 1, wherein the first and second suture clamp pads are proud relative to the first and second needle clamp pads, respectively, when the first and second jaw members are in each of the open state and the first closed state.

10. The apparatus of claim 1, wherein the first and second suture clamp pads are at least one of flush with or recessed relative to the first and second needle clamp pads, respectively, when the first and second jaw members are in the second closed state.

11. The apparatus of claim 1, wherein the first and second suture clamp pads are substantially surrounded by respective portions of the first and second needle clamp pads, respectively.

12. The apparatus of claim 1, wherein the first and second needle clamp pads include first and second recesses, respectively, wherein the first and second suture clamp pads are securely retained within the first and second recesses, respectively.

13. The apparatus of claim 1, wherein the first and second needle clamp pads each include a plurality of rigid teeth.

14. The apparatus of claim 1, wherein the first and second suture clamp pads each include a plurality of flexible protrusions.

15. The apparatus of claim 1, wherein the first closed state is a partially closed state, wherein the second closed state is a fully closed state.

16. An apparatus, comprising:
(a) a shaft extending along a longitudinal axis to a distal end; and
(b) an end effector operatively coupled to the distal end of the shaft, the end effector including a pair of jaw members, the pair of jaw members being pivotable relative to each other for gripping at least one surgical object, at least one jaw member of the pair of jaw members including:
(i) a first clamp pad, and
(ii) a second clamp pad configured to transition between an undeformed state and a deformed state, the second clamp pad of the at least one jaw member extending laterally inwardly relative to the respective first clamp pad at least when in the undeformed state.

17. The apparatus of claim 16, wherein the first clamp pad of the at least one jaw member is rigid.

18. The apparatus of claim 16, further comprising a wrist, wherein the end effector is operatively coupled to the distal end of the shaft via the wrist.

19. A system, comprising:
(a) an end effector configured to transition between a suture-gripping mode in which the end effector is configured to apply a soft grip to a suture, and a needle-gripping mode in which the end effector is configured to apply a hard grip to a needle; and
(b) a controller configured to transition the end effector between the suture-gripping mode and the needle-gripping mode, wherein the controller is configured to provide at least one of audible feedback or haptic feedback to indicate transitioning of the end effector from the suture-gripping mode to the needle-gripping mode.

20. The system of claim 19, wherein the controller includes at least one detent configured to generate the at least one of audible feedback or haptic feedback during transitioning of the end effector between the suture-gripping mode and the needle-gripping mode.
